(19) United States
(12) Patent Application Publication
(10) Pub. No.: US 2015/0157295 A1
(43) Pub. Date: Jun. 11, 2015

(54) ULTRASOUND SYSTEM AND POINTER CONTROLLER

(71) Applicant: QISDA CORPORATION, Taoyuan (TW)

(72) Inventors: Fang-Bin Liu, Hsinchu City (TW); Shih-Yu Lee, Taichung City (TW)

(73) Assignee: QISDA CORPORATION, Taoyuan (TW)

(21) Appl. No.: 14/100,026

(22) Filed: Dec. 9, 2013

(51) Int. Cl. A61B 8/00; A61B 8/14

(52) U.S. Cl. CPC A61B 8/465; A61B 8/4444; A61B 8/463; A61B 8/14; A61B 8/54; A61B 8/467

(57) ABSTRACT

The present invention provides an ultrasound system comprises a display and a pointer controller. The pointer controller has a probe head, a control unit and a probe body. The display displays a pointer and an ultrasound image according the ultrasound signals received by the probe head. When pushing a button of the probe body or handling the probe body in a specific manner, the control unit switches the probe head to control the pointer on the display from the scanning the ultrasound image.

This application claims the benefit of People's Republic of China application Serial No. 201210525298.9, filed Dec. 10, 2012, the disclosure of which is incorporated by reference herein in its entirety.

### BACKGROUND OF THE INVENTION

1. Field of the Invention

The present invention relates to a pointer controller and an ultrasound system, and particularly to a probe of an ultrasound system that can switch functions between controlling a pointer and scanning an ultrasound image.

2. Description of the Prior Art

In the prior art, when a user wants to use an ultrasound system to execute a ultrasound scanning function by a probe, the user needs one of his hands to hold the probe and the other hand operating the user interface on the display of the ultrasound system for operating ultrasound images functions, for example to save image, zoom in/out image etc. The user interface can be a touched panel, a keyboard, buttons or a pointer controller. The pointer controller controls a pointer, or a cursor, on the display for controlling the GUI. Generally, the pointer controller is a mouse or a tracking ball. Sometimes, when the user holds the probe with one hand and the other hand needs to aid and comforts the patient who is serious injured. In that case, the user has difficulties to operate the probe and the mouse, or a tracking ball, in the same time. It would cause user inconvenient in operating the ultrasound system.

Therefore, how to make the user operate the ultrasound system through the probe and the user interface of the ultrasound system more easily becomes an important issue for a designer.

### SUMMARY OF THE INVENTION

This invention provides a pointer controller and an ultrasound system which can be easily switching functions between controlling a pointer on the display and scanning an ultrasound image.

The present invention provides an ultrasound system comprises a display and a pointer controller. The pointer controller has a probe head, a control unit and a probe body. When the probe head put on the skin of a human body, the probe head transmits and receives ultrasound signals and the ultrasound system calculating the received ultrasound signals to generate an ultrasound image displaying on the display. The display also displays a pointer and operating buttons for handling the ultrasound image processing, for example saving, zoom in/out or editing the image. The pointer is controlled by user interfaces of the ultrasound system, for example a mouse or a tracking ball. In this invention, the user can handle the probe body in a specific way to activate the control unit switches the probe head controlling the pointer on the display from scanning the ultrasound image.

The probe body can arrange a button on its surface. When the user handles the probe body and pushed the button. The control unit switches the probe head to control the pointer on the display. In the mean time, the display displays a frozen ultrasound image. When the probe head moves on the skin, the pointer moves on the display accordingly. The control unit can estimated a movement of the probe head according the reflected ultrasound signals or the change of the ultrasound image, the control unit making a movement indicator for the display to move the pointer accordingly.

When the probe head working on the ultrasound scanning mode, the control unit can also detecting whether the probe head is moving in a specific manner. In case, the probe body is handled to let the probe head's moving manner match a predetermined manner, the control unit switches the probe head to control the pointer on the display. The specific manner of the probe head can be the probe head moving to predetermined directions in sequence during a predetermined time or the probe head moving forward and backward in the first and second directions in a predetermined range.

When the probe head controls the pointer on the display, the display may displaying menu buttons, when the probe head controls the pointer and the pointer moves on one of the buttons displaying on the display at least staying for a predetermined time, the control unit selecting the button.

When the probe head controls the pointer one the display, when the probe head moving in a first manner, the control unit executing a related function of the selected button. The first manner can be the probe head moving forward and backward in the first and second directions in a predetermined range. The predetermined range can be determined from 0.5 to 2 centimeter.

The first manner can also be the probe head moving to predetermined directions in sequence during a predetermined time. The predetermined time can be equal or less than 0.5 second.

When the probe head moving in a second manner, the control unit canceled the selection of the button. The second manner can be set similar but different to the first manner.

The probe body can set a light transceiver, when the probe head controlling the pointer on the display, the light transceiver transmitting light and receiving reflected light to determine a movement of the pointer on the display. That's said, the probe head may stop transmitting the ultrasound and works as a mouse by using the light transceiver.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is the first embodiment of the pointer controller.

FIG. 2 is the second embodiment of the pointer controller.

### DETAILED DESCRIPTION

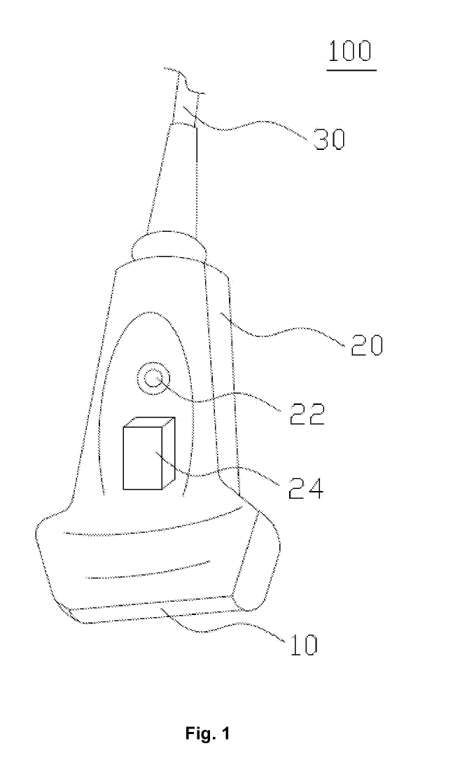

Please refer to FIG. 1, pointer controller 100 connects to an ultrasound system for transmitting and receiving signals. The ultrasound system has a display for displaying a ultrasound image according to the signals. Similar structure can refer to U.S. pat. Nos. 8,517,946 For 6,685,637.

Pointer controller 100 comprises probe head 10, probe body 20 and a control unit set in probe body 20. Pointer controller 100 electrically connects to an ultrasound system by connecting wire 30. Probe head 10 generates ultrasound signals for transmitting and receiving ultrasound signals reflected from the human body. Push button 22 sets on probe body 20 and electrically connects the control units. Push button 22 can be made by waterproof materials for using in different conditions.

When the user handles probe body 20 moving on a human body, the display displays an ultra image according the ultrasound signals. The display can also display menu buttons and a pointer for the user easily operating the ultrasound system or processing the image. When pushing push button 22, the control unit switches probe head 10 to control the pointer on the display. Probe head 10 may transmit and receive ultrasound signals and moving on the skin of human body. The control unit can estimate direction and movements of probe head according to the ultrasound signals. The pointer can be moved based on the above estimated direction and movements. The user doesn't need to use a mouse or a tracking ball, to move the pointer. If user pushed push button 22 again, probe head 10 comes back the original function for scanning ultrasound images.

When probe head 10 controls the pointer on the display, the user may make a long push by push button 22, for example 2 seconds. The display pops up a menu with operating buttons, each of the buttons related to at least one function, for example to save image, zoom in/out image etc. The user may move the pointer to any one of the button by moving probe head 10 on the skin of human body. If the pointer stayed on the button more than a predetermined period, for example 2 second, the button is selected. If probe head 10 was moved in a first manner, the related function of the selected button is executed. If probe head 10 was moved in a second manner, the selected button is canceled. For carefully executed or selected/deselected buttons, an optional step may be added, after the action, the display may display “Yes” and “No” buttons for further confirmations. The user may move the pointer on “Yes” or “No” buttons and moving by a third manner as a final confirmation. The first, second and third manner can be probe head 10 moving in a specific way. For example, probe head 10 moves forward and backward in the first direction and the second direction in a predetermined period. The predetermined range is selectively in 0.5 to 2 centimeter. Or probe head 10 moves in predetermined directions in sequence in a predetermined time different directions, for example, the pointer moves in first direction and then second direction and then the third direction in 0.5 second. The first, second and third manner can be defined as the above examples or they can be defined by the user.

When probe head 10 working as an ultrasound probe, the control unit can estimate directions and movements of probe head 10 according to the ultrasound signals in the same time. The control unit tracks the moving manner of probe head 10. In case the moving manner matches a specific manner, the control unit switches probe head 10 to control the pointer on the display. The specific manner can be selected from any of the first to the third manners or defined by the system designer.

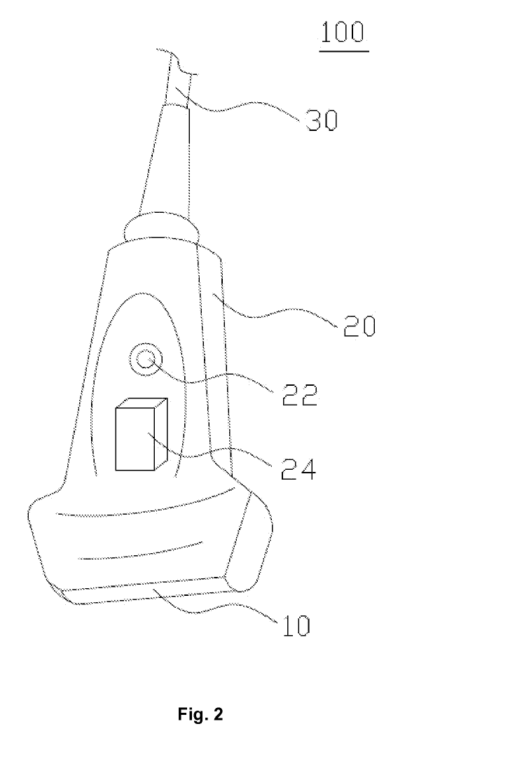

Please refer to FIG. 2, pointer controller 100 comprises probe head 10, probe body 20 and a control unit set in probe body 20. Pointer controller 100 electrically connects to an ultrasound system by connecting wire 30. Probe body 20 further comprises light transmitter 24. Light transmitter 24 can emits light and received the reflected light. When pushing push button 22, the control unit switches probe head 10 to control the pointer on the display. The control unit can estimated a movement of probe head 10 according the reflected light. In other words, probe head 20 works as an optical computer mouse. In this case, the ultrasound signals may stop transmitting after push button 22 pushed. As probe head 20 works as an optical computer mouse, probe head 20 can move in different surface, for example on the surface of a table or a mouse pad.

To sum up, in this invention, user may control the pointer by handling probe body 20. That would make user use ultrasound system by one hand, and the other hand can aid the patient in the same time. That would be more convenient.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A pointer controller connecting to an ultrasound system, the ultrasound system having a display, the pointer controller comprising:
a probe head for transmitting and receiving ultrasound signals, the display displaying a pointer and an ultrasound image according to the ultrasound signals;
a control unit; and
a probe body having a button connecting the control unit, when the button pushed, the control unit switching the probe head to control the pointer on the display.

2. The pointer controller of claim 1, when the probe head controlling the pointer on the display, the control unit determining a movement for the pointer moving on the display according the ultrasound signals.

3. The pointer controller of claim 1, the display displaying menu buttons, when the probe head controlling the pointer and moving the pointer on one of the buttons displaying on the display, the control unit selecting the button.

4. The pointer controller of claim 3, when the probe head moving in a first manner, the control unit executing a related function of the selected button.

5. The pointer controller of claim 4, the probe head moving forward and backward in a first and second directions in a predetermined ranges as the first manner.

6. The pointer controller of claim 5, the predetermined range selectively being in 0.5 to 2 centimeter.

7. The pointer controller of claim 4, the probe head moving to predetermined directions in sequence in a predetermined time as the first manner.

8. The pointer controller of claim 4, the predetermined time is equal or less than 0.5 second.

9. The pointer controller of claim 3, when the probe head moving in a second manner, the control unit canceled the selection of the button.

10. The pointer controller of claim 1, the probe body further comprising a light transceiver, when the probe head controlling the pointer on the display, the light transceiver transmitting light and receiving reflected light to determine a movement of the pointer on the display.

11. An ultrasound system comprising:
a pointer controller comprising a probe head, a control unit and a probe body, the probe head transmitting and receiving ultrasound signals;
a display displaying a pointer and an ultrasound image according to the ultrasound signals, the control unit switching the probe head to control the pointer on the display from the scanning the ultrasound image by handling the probe body.

12. The ultrasound system of claim 11, the probe body comprising a button, when the button pushed, the control unit switching the probe head to control the pointer on the display.

13. The ultrasound system of claim 11, when the probe head controlling the pointer on the display, the control unit determining a movement for the pointer moving on the display according the ultrasound signals.

14. The ultrasound system of claim 11, the display displaying menu buttons, when the probe head controlling the pointer and moving the pointer on one of the buttons, the control unit selecting the button.

15. The ultrasound system of claim 14, when the probe head moving in a first manner, the control unit executing a related function of the selected button.

16. The ultrasound system of claim 15, the probe head moving forward and backward in a first and second directions in a predetermined ranges as the first manner.

17. The ultrasound system of claim 11, when the control unit detecting the probe head moving in a third manner, the control unit switching the probe head to control the pointer on the display.

18. The ultrasound system of claim 17, the control unit detecting the third manner according the ultrasound signals.

19. The pointer controller of claim 17, the probe head moving to predetermined directions in sequence in a predetermined time as the third manner.

20. The pointer controller of claim 17, the probe head moving forward and backward in a first and second directions in a predetermined range as the third manner.
